(19) United States
(12) Patent Application Publication
(10) Pub. No.: US 2025/0166812 A1
(43) Pub. Date: May 22, 2025

(54) CAREGIVER COMMUNICATIONS DEVICE AND METHOD

(71) Applicant: Brian SKUSE, Boston, MA (US)

(72) Inventor: Brian SKUSE, Boston, MA (US)

(21) Appl. No.: 18/956,753

(22) Filed: Nov. 22, 2024

Related U.S. Application Data
(60) Provisional application No. 63/602,154, filed on Nov. 22, 2023.

(51) Int. Cl. G16H 40/63; H04W 4/80; H04W 84/12

(52) U.S. Cl. CPC G16H 40/63; H04W 4/80; H04W 84/12

(57) ABSTRACT

A simplified communications device includes a housing; press or touch keys coupled to the housing, each key corresponding to a different status message of a care recipient; a first memory situated within the housing and storing a first application; a processor situated within the housing, the processor configured to, in conjunction with the first application, cause the first application on the first computing device to send a status message corresponding to one of the plurality of keys, wherein the simplified communications device is capable of a wireless connection to the first computing device associated with the care recipient; and based on one of the keys being pressed, the status message is automatically sent from the first application executing on the first computing device to a second application executing on a second computing device associated with a caregiver.

### FIELD OF THE INVENTION

The present invention generally relates to communication devices and more specifically to a simplified caregiver communications device with multiple keys, each key corresponding to a different status message, the communications device having a limited purpose of conveying a status of a care recipient.

### BACKGROUND OF THE INVENTION

Caregiver burnout is a significant and growing issue, with many caregivers experiencing physical, emotional, and financial strain due to their responsibilities. This has a negative impact on their well-being and the quality of care they provide. Caregiver burnout has been described as a state of physical, emotional and mental exhaustion and those experiencing caregiver burnout may experience fatigue, anxiety and depression.

The numbers put caregiving in perspective. In 2020, 41.8 million Americans provided unpaid care to an adult over the age of 50. Nearly 17% of the U.S. adult population. 89% of caregivers provide care for a relative or other loved one, such as a spouse. 23.7 hours per week is the average amount of time caregivers spend providing unpaid care for loved ones they don't live with; those who live with their care recipient spend 37.4 hours a week. More than 75% of all caregivers are female. The average caregiver is 50.1 years old. Caregivers provide an estimated $470 billion in free labor each year. 89% of unpaid caregivers are related to their care recipients by either blood or marriage. 11% care for a friend, neighbor, or another non-relative, 48% care for a parent, 11% care for a spouse or long-term partner, 10% care for a grandparent or grandparent-in-law, 9% care for a parent-in-law, 5% care for a sibling or sibling-in-law, 5% care for another relative and 1% of caregivers of people over age 50 care for their adult child.

Common reasons for in-home caregiving include 16% “old age” or general infirmity, 12% mobility assistance, 11%, Alzheimer's disease or other types of dementia, 6% cancer care, 8% post-surgery or wound care, 5% mental illness or emotional inability to care for themselves, 5% post-stroke care, 4% diabetes care, 4% fall risk and 3% blindness or vision loss.

In the past, caregiver communication products have typically been limited to a single, basic “call button,” where the care recipient presses a single button to alert the caregiver. However, this approach lacks details about the nature of the alert and the corresponding urgency of the of the status message. The invention offers a more nuanced messaging system that allows caregivers to better understand the care recipient's needs before responding. For example, requests like “pain” or “water” carry varying degrees of urgency and stress for caregivers, unlike a traditional “help” button, which is always the same. The invention can reduce unnecessary over communication between the caregiver and care recipient. This is accomplished with the simplified communications device of the present invention.

The invention helps streamline caregiving tasks by providing real-time requests from the care recipient when they need it providing greater independence to the care recipient and more autonomy to the caregiver. By easing the challenges of caregiving, the invention will improve the lives of both caregivers and care recipients. Caregivers often face challenges in understanding the immediate needs of those they care for, especially when verbal communication is limited. The invention can provide a direct and clear way for the person receiving care to communicate their needs, reducing frustration and misunderstandings. Caregivers can also quickly respond to the requests made, providing timely assistance. This can prevent unnecessary discomfort and improve the overall care experience.

Caregivers often worry about missing important cues or needs of the one receiving care. The invention can ease this anxiety by offering a structured and reliable method for the person to express their needs. The invention can give the person receiving care a sense of control and agency over their needs. This empowerment can contribute to improved emotional well-being and a more positive care relationship. By receiving specific requests, caregivers can allocate their time and resources more efficiently. This prevents caregivers from constantly checking in on the person's needs and allows them to focus on their caregiving tasks. The invention can also help in reducing the constant demand for attention, as caregivers can address needs in a more organized and balanced manner. This, in turn, can decrease stress and emotional exhaustion. By streamlining communication and response processes, caregivers can find more time for self-care, which is crucial for preventing burnout and maintaining their own well-being.

There thus exists a need to help address the serious problem of caregiver burnout.

### SUMMARY OF THE INVENTION

The invention addresses the above-referenced need by providing, in a first aspect, a simplified communications device, comprising: a housing; a plurality of keys coupled to the housing, each key corresponding to a different status message of a care recipient; a first memory situated within the housing and storing a first application; a processor situated within the housing, the processor configured to, in conjunction with the first application, cause the first application on the first computing device to send a status message corresponding to one of the plurality of keys, wherein the simplified communications device is capable of a wireless connection to the first computing device associated with the care recipient; and wherein based on one of the plurality of keys being pressed, the status message is automatically sent from the first application executing on the first computing device to a second application executing on a second computing device associated with a caregiver.

In a second aspect, the invention comprises a method of communicating a status of a person, the method comprising: providing a simplified communications device and a first application to a care recipient, wherein the simplified communications device comprises: a housing; a plurality of keys coupled to the housing, each key corresponding to a different status message of a care recipient; a first memory situated within the housing and storing the first application; and a processor situated within the housing, the processor configured to, in conjunction with the first application, cause the first application on the first computing device to send a status message corresponding to one of the plurality of keys, wherein the simplified communications device is capable of a wireless connection to the first computing device associated with the care recipient; providing a second application stored in a second memory associated with a second computing device associated with a caregiver; and based on one of the plurality of keys being pressed, automatically sending a status message corresponding to the one of the plurality of keys from the first application executing on the first computing device to the second application executing on the second computing device.

Numerous applications, some examples of which are described below, may be implemented using the present invention.

### BRIEF DESCRIPTION OF THE DRAWINGS

The above-mentioned and other features and advantages of this invention, and the manner of attaining them, will become apparent and be better understood by reference to the following description of the invention in conjunction with the accompanying figures, wherein:

FIG. 1 depicts one example of a simplified communications device, in accordance with one or more aspects of the present invention;

FIG. 2 is a flow diagram for one example of a method of communicating a status of a care recipient using the simplified communications device of FIG. 1, in accordance with one or more aspects of the present invention;

FIG. 3 depicts one example of requesting an application for the simplified communications device of FIG. 1 by a care recipient, in accordance with one or more aspects of the present invention;

FIG. 4 depicts one example of the care recipient of FIG. 3 pressing one of the keys on the simplified communications device, in accordance with one or more aspects of the present invention;

FIG. 5 depicts one example of a caregiver receiving on their computing device a status of the care recipient in response to the care recipient pressing the key of FIG. 4, in accordance with one or more aspects of the present invention; and

FIG. 6 depicts one example of a computing device, in accordance with one or more aspects of the present invention.

FIG. 7 depicts none example of a printed circuit board assembly for the simplified communications device of FIG. 1, in accordance with one or more aspects of the present invention.

Corresponding reference characters indicate corresponding parts throughout the several views. The embodiments set out herein are examples only and illustrate currently preferred embodiments of the present invention, and such examples are not to be construed as limiting the scope of the invention in any manner.

### DETAILED DESCRIPTION

As used herein, a “simplified communications device” refers to a limited-purpose electronic communications device with two or more keys, each of the keys corresponding to a different status message of a person using the simplified communications device, the simplified communications device relying on a wirelessly connected computing device and an application on the computing device to automatically send a status message when one of the keys is pressed.

As used herein, the term “key” or “keys” refers to keys, buttons or the like on a simplified communications device, for example, one or more capacitive touch screens, or, as another example, physical keys that can be pushed and doing so causes a signal indicative of a status of a care recipient to be sent out from the simplified communication device to a computing device associated with the care recipient.

As used herein, the term “computing device” or “computing unit” refers to a computing device with one or more processors, memory and one or more applications stored in the memory. For example, the applications can include an operating system, a photo application, messaging application and/or other general or special purpose applications. Examples of computing devices include desktop computers, laptop computers, smart phones, tablets, tablet computers, small form factor computers, etc. In addition, such computing devices may be accompanied by one or more peripheral devices. For example, peripheral devices may include a pen or other pointing device, a keyboard, a monitor, etc.

As used herein, the term “processor” includes “chips,” central processing units (CPUs), graphics processing units (GPUs), integrated circuitry performing processing (e.g., field programmable gate arrays (FPGAs), integrated circuitry made using large-scale integration (LSI) or very large-scale integration (VLSI)), logic circuits and “systems on chip,” which include most or all of the components of a computer. Processors work in conjunction with applications, apps, software, firmware and the like, and may also work in conjunction with other hardware, to accomplish programmed tasks.

As used herein, the term “short-range wireless communications protocol” refers to protocols for wireless communication at distances of up to about 10 meters, though one or more on the following list may go further in practice. In addition, new or improved forms of short-range wireless communications protocols may extend the range. Examples include Bluetooth, Bluetooth Low Energy, Wi-Fi, a combination of low-power connectivity and a high-speed connection, near-field communication (NFC), radio frequency identification (RFID), low-power wide area network (LPWAN), ultra-wideband (UWB) and low-rate wireless personal area network (LR-WPAN).

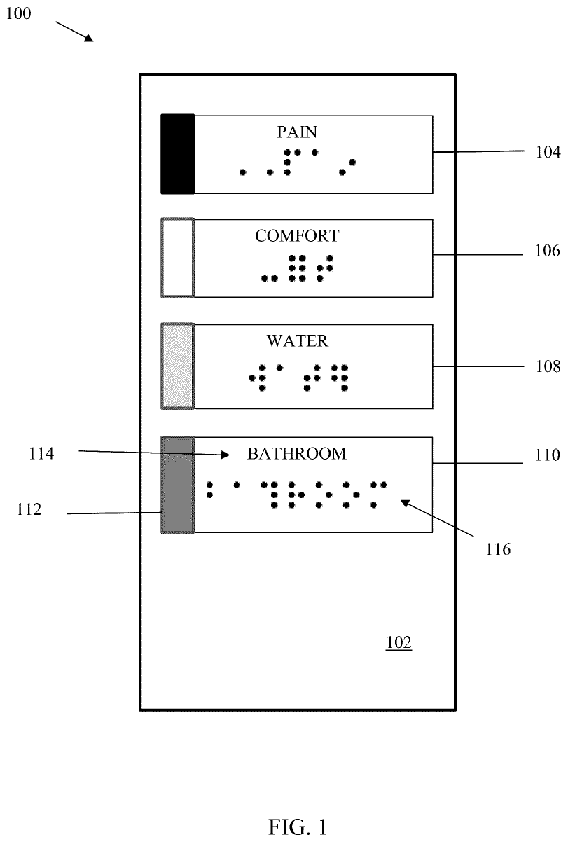

Referring now to the drawing, FIG. 1 depicts one example of a simplified communications device 100, in accordance with one or more aspects of the present invention. The simplified communications device 100 may include, for example, a housing 102 and four keys 104, 106, 108 and 110. Although the example of FIG. 1 includes four keys, it will be understood that there could be as few as two keys or more than four keys. Each key corresponds to a different status message of a care recipient. The status messages in this example include, “pain,” “comfort,” “water” and “bathroom,” each message corresponding to a key 104, 106, 108 and 110, respectively.

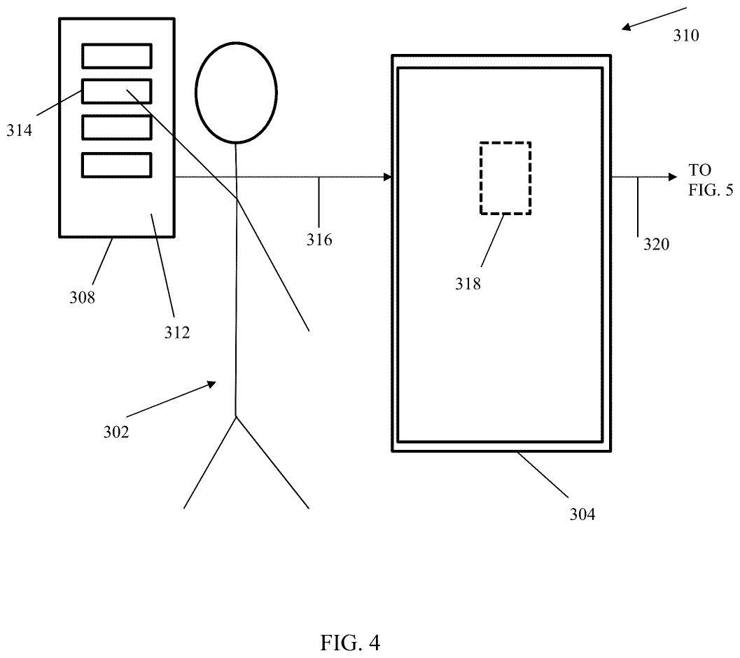

In one embodiment, each key includes one or more forms of a descriptor thereon or in close proximity thereto, the descriptor indicating the corresponding message. For example, key 110 includes a color or grayscale 112 different from colors or grayscale of the other keys. As another example of a message descriptor, text 114 may be included. As still another example, the message descriptor(s) may include raised Braille characters 116. In one example, the keys may include one or more touch screens (e.g., a single touch screen including areas thereof for each of the keys, or four separate, smaller touch screens). In one example, the one or more touch screens may include haptic feedback or another form of feedback. In another example, the keys may include physical keys, preferably physical keys that “click” to provide feedback. Preferably, the keys are spaced apart for ease of use and to help ensure two or more adjacent keys are not pressed together. In one example, there are four keys 104, 106, 108 and 110 spaced apart from each other on the simplified communications device 308 in a vertical column, as in FIG. 4.

In some cases, the person receiving care will be mobile and will carry or attach a lanyard to the simplified communications device. In other cases, the person receiving care may not be mobile and may be in a bed. In that case, the person receiving care will have the simplified communications device on the bed or a nightstand or tray. Alternatively, the simplified communications device could be attached to something stationary in close proximity to the person receiving care, preferably a removable attachment, to, for example, a side rail of the bed.

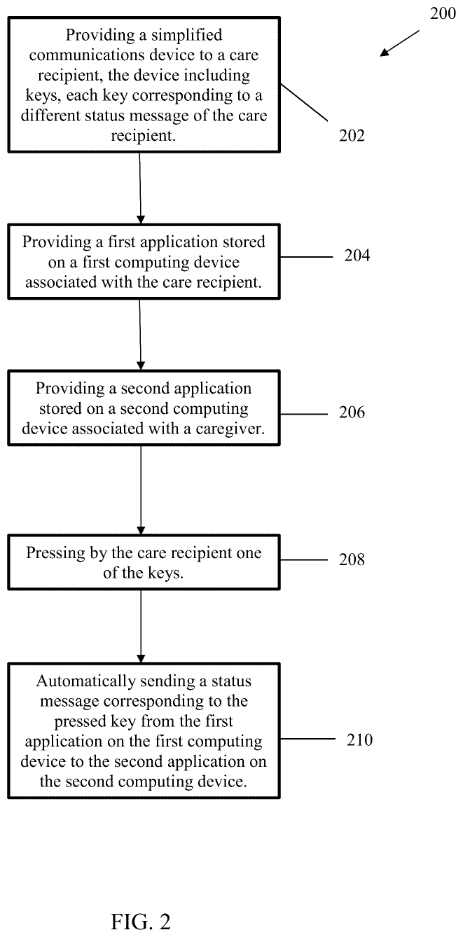

FIG. 2 is a flow diagram 200 for one example of a method of communicating a status of a care recipient using the simplified communications device of FIG. 1, in accordance with one or more aspects of the present invention. The method may include, for example, providing a simplified communications device to a care recipient, the simplified communications device including at least two keys, each key corresponding to a different status message of the care recipient, step 202. The method may also include, for example, providing a first application stored on a first computing device associated with the care recipient, step 204. The method may further include, for example, providing a second application stored in a memory of a second computing device associated with a caregiver, step 206. The method may also include, for example, pressing by the care recipient one of the keys of the simplified communications device, step 208. The method may further include, for example, automatically sending a status message corresponding to the pressed key from the first application on the first computing device to the second application on the second computing device, step 210.

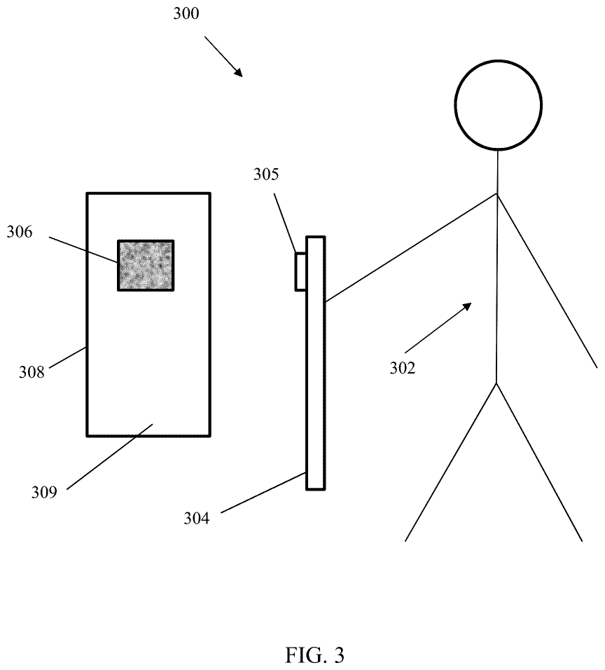

FIGS. 3-5 depict examples of various stages of performing the method of the present invention. FIG. 3 depicts, not to scale for ease of viewing, one example of requesting an application 300 for a simplified communications device 308 by a care recipient 302, in accordance with one or more aspects of the present invention. For example, the requesting may include requesting an application for the simplified communications device from a manufacturer of the simplified communications device or a business entity associated with providing a status message service. The care recipient 302 may, for example, use a first computing device 304 associated with the care recipient 302 to perform steps of the method. The first computing device 304 may be owned, rented, leased by or lent to the care recipient 302. For example, the first computing device 304 may be part of the subscription service or simply owned by the care recipient 302. In this regard, the care recipient 302 may use an imaging sensor 305 on the first computing device 304 (e.g., via a photo application or dedicated QR code reader application) to scan, for example, a “QR” code 306 on a simplified communications device 308 of the present invention. As one skilled in the art will know, a QR code (short for “quick-response code”) is a type of two-dimensional matrix barcode. In one example, the QR code 306 may be placed, for example, on a back side 309 of the simplified communications device 308, while the keys may be placed, for example, on a front side thereof (312, FIG. 4). Other possible methods to request an application for the simplified communications device 308 include, for example, calling a call center associated with the business entity, having a video chat with a customer service representative, manually requesting an application for the simplified communications device 308 online, e.g., on a web site associated with the business entity or via email or text messaging.

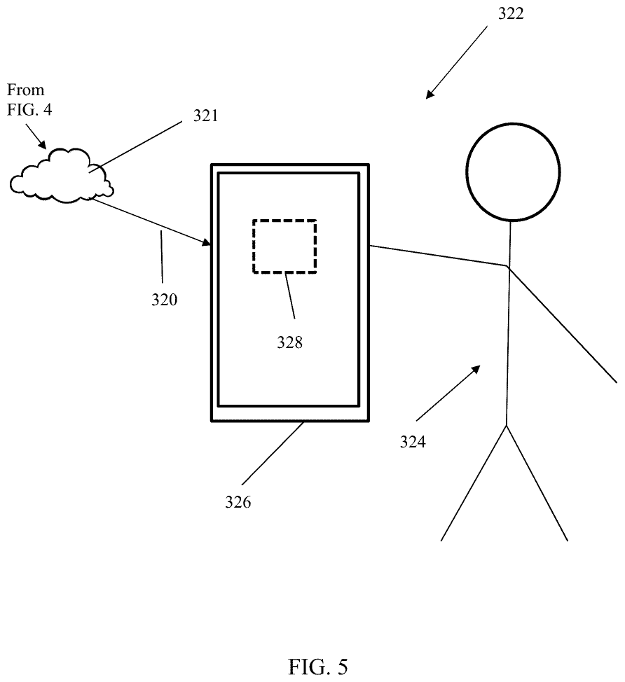

FIG. 4 depicts, not to scale for ease of viewing, one example 310 of the care recipient 302 of FIG. 3 pressing one of the keys (e.g., key 314) on the simplified communications device 308, in accordance with one or more aspects of the present invention. Based on one of the keys being pressed, a signal 316 is sent from the simplified communications device 308 to an application 318 executing on the first computing device 304. Based on receipt of the signal 316, for example, over a network (321, FIG. 5) (e.g., the Internet, a private network, personal area network or a cellular network) from the simplified communications device 308, a status message 320 of the care recipient corresponding to the key 314 pressed may be, for example, automatically sent from the application 318 executing on the first computing device 304 to a second application (328, FIG. 5) executing on the second computing device (326, FIG. 5).

FIG. 5 depicts, not to scale for ease of viewing, one example 322 of a caregiver 324 receiving on a second computing device 326 associated with the caregiver, a status of the care recipient 302 in response to the care recipient 302 pressing the key 314 of FIG. 4, in accordance with one or more aspects of the present invention. The status message 320 is then received by the second application 328 executing on the second computing device 326. At that point, the caregiver may act on the status message by, for example, physically checking on the care recipient, calling the care recipient, emailing or texting the care recipient. As another example, the caregiver may forward the status message to another family member or colleague, or to a team of caregivers for the care recipient.

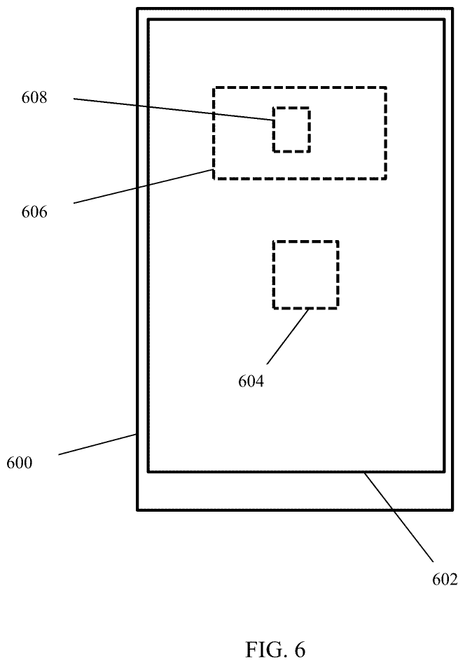

FIG. 6 depicts one example of a computing device 600, in accordance with one or more aspects of the present invention. The computing device includes, for example, a screen 602 that may or may not be bendable or foldable, one or more processors 604 and memory 606 in which is stored one or more applications 608, for example, an operating system, photo application or other general or special purpose applications. Examples of memory include, but are not limited to, random access memory (RAM), dynamic random access memory (DRAM), solid state memory, read only memory (ROM), programmable ROM, erasable programmable ROM, electrically erasable programmable ROM, flash memory, cache memory, double data rate synchronous DRAM (DDR), volatile memory, double data rate 4 synchronous dynamic RAM (DDR4 SDRAM), double data rate 2 synchronous dynamic RAM (DDR2), double data rate 3 synchronous dynamic RAM (DDR3), static RAM and erasable programmable ROM (EPROM).

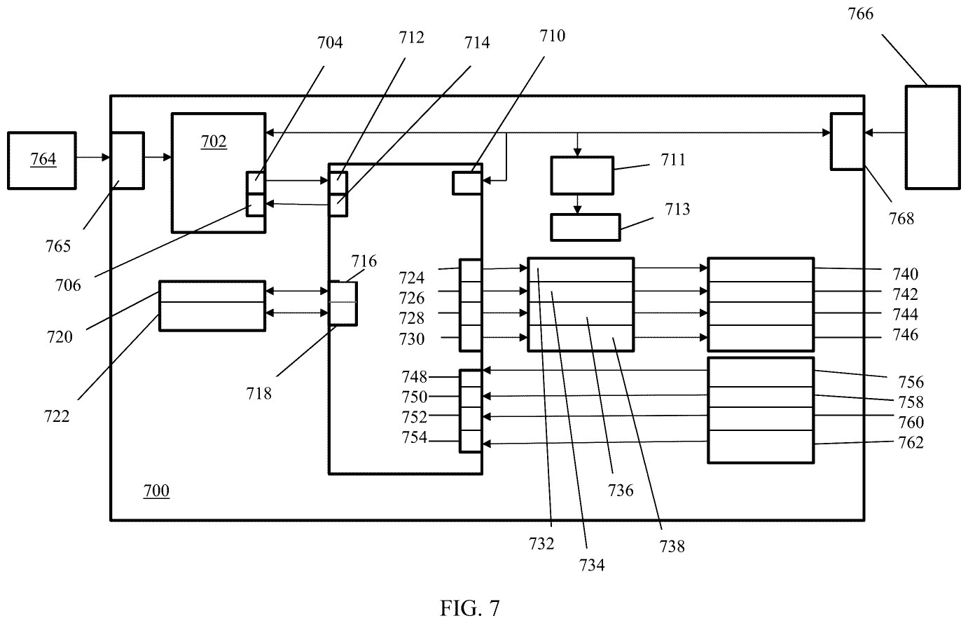

FIG. 7 depicts one example of a printed circuit board assembly 700 (“PCBA”) for the simplified communications device 100 of FIG. 1, in accordance with one or more aspects of the present invention. The PCBA may include, for example, a wireless power battery charger 702, having an output 704 (e.g., a Schmitt Trigger or “ST”) and an input 706 (e.g., an Energy Note or “EN”). One example of a commercially available wireless power battery charger is model BQ5105xB from Mouser Electronics, Inc. in Mansfield, Texas. Also included in the PCBA 700 is a multiprotocol module 708; in this case, having Bluetooth and Wi-Fi protocols. One example of a commercially available multiprotocol module is ESP-C6-MINI-1 from Espressif Systems located in Shanghai, China. Multiprotocol module 708 may include, for example, an analog-to-digital converter 710, input 712 and output 714. The analog to digital converter 710 may have a bidirectional connection to a buck regulator 711 and VCC 713 (about 3.6 volts). Multiprotocol module 708 may further include, for example, input/output 716 and 718, each with bidirectional capability, for JTAG 720 and UART 722, respectively. As one skilled in the art will know, “JTAG” stands for joint test action group. JTAG is an integrated method for testing interconnects on printed circuit boards (PCBs) that are implemented at the integrated circuit (IC) level and “UART” stands for universal asynchronous receiver/transmitter and defines a protocol, or set of rules, for exchanging serial data between two devices. Also included in multiprotocol module 708 are four outputs 724, 726, 728 and 730, each driving an LED driver 732, 734, 736 and 738, respectively. The LED drivers 732, 734, 736 and 738 control LEDs 740, 742, 744 and 746, respectively. The multiprotocol module 708 may further include, for example, inputs 748, 750, 752 and 754 for input from keys 756, 758, 760 and 762, respectively, electrically connected to keys 104, 106, 108 and 110 in the simplified communications device 100 of FIG. 1. In addition, to facilitate wireless charging is a charging coil 764 (e.g., 25 watts) to charge through connector 765 and battery 766 (e.g., a lithium polymer battery of 2000 mAh capacity) via connector 768.

In a first aspect, the invention includes a simplified communications device. In one embodiment, the simplified communications device may include, for example, a housing and keys coupled to the housing, each key corresponding to a different status message of a care recipient. The simplified communication device may further include, for example, a first memory situated within the housing and storing a first application and may also include, for example, a processor situated within the housing, the processor configured to, in conjunction with the first application, cause the first application on the first computing device to send a status message corresponding to one of the keys, the simplified communications device being capable of a wireless connection to the first computing device associated with the care recipient; and based on one of the plurality of keys being pressed, the status message is automatically sent from the first application executing on the first computing device to a second application executing on a second computing device associated with a caregiver.

In another example, the wireless connection of the simplified communications device of the first aspect may use, for example, a short-range wireless communications protocol. As examples, the short-range wireless communications protocol may include, for example, one of Bluetooth, Wi-Fi, a combination of low-power connectivity and a high-speed connection, near-field communication (NFC), radio frequency identification (RFID), low-power wide area network (LPWAN), ultra-wideband (UWB) and low-rate wireless personal area network (LR-WPAN).

In still another example, the first application and the second application of the first and second computing devices, respectively, may be, for example, a same application.

In yet another example, the second application may be stored in a second memory associated with the second computing device. In one example, each of the first memory and the second memory may include one of an integrated memory and a removable memory (e.g., a flash memory device).

In another example, the simplified communications device of the first aspect may further include one or more descriptors indicating status messages associated with the keys. For example, the descriptor may take the form of at least one of a different color or grayscale for each of the plurality of keys, descriptive Braille on or adjacent to each of the plurality of keys and descriptive text on or adjacent to each of the keys.

In still another example, the keys may include, for example, physical keys.

In yet another example, the keys of the simplified communications device of the first aspect may include, for example, one or more capacitive touch screens with or without haptic or other feedback, such as, for example, an LED light, a blinking LED light, various colors of LED lighting or blinking LED lighting of various colors.

In a second aspect, the invention includes a method of communicating a status of a care recipient. The method may include, for example, providing a simplified communications device and a first application to the care recipient, the first application being made available, for example, by downloading the same. The simplified communications device may include, for example, a housing and keys coupled to the housing, each key corresponding to a different status message of a care recipient. The simplified communications device may further include, for example, a first memory situated within the housing and storing the first application and may also include, for example, a processor situated within the housing, the processor configured to, in conjunction with the first application, cause the first application on the first computing device to send a status message corresponding to one of the keys, the simplified communications device being capable of a wireless connection to the first computing device associated with the care recipient. The method may further include, for example, providing a second application, for example, by making it available for download, stored in a second memory associated with a second computing device associated with a caregiver and based on one of the plurality of keys being pressed, automatically sending a status message corresponding to the one of the plurality of keys from the first application executing on the first computing device to the second application executing on the second computing device.

In one example, the keys of the simplified communications device in the method of the second aspect may include two or more keys.

In another example, the wireless connection of the simplified communications device in the method of the second aspect may use, for example, a short-range wireless communications protocol. In examples, the short-range wireless communications protocol may include, for example, one of Bluetooth, Wi-Fi, a combination of low-power connectivity and a high-speed connection, near-field communication (NFC), radio frequency identification (RFID), low-power wide area network (LPWAN), ultra-wideband (UWB) and low-rate wireless personal area network (LR-WPAN).

In still another example, the first application and the second application of the simplified communications device may be, for example, a same application. In another example, where the same application is provided to both the care recipient and the caregiver, the application may look and/or function differently for each person in accordance with their roles.

In yet another example, in the method of the second aspect, the second application may be stored, for example, in a second memory associated with the second computing device. In one example, each of the first memory and the second memory may include one of an integrated memory and a removable memory (e.g., a flash memory device).

In another example, the simplified communications device of the first aspect may further include at least one of: a different color or grayscale for each of the keys; descriptive Braille on or adjacent to each of the plurality of keys and descriptive text on or adjacent to each of the keys.

In yet another example, the keys of the simplified communications device of the method of the second aspect may include, for example, one or more capacitive touch screens with or without haptic feedback or other feedback, such as, for example, one or more different sounds, an LED light, blinking LED light, various colors of LED lighting or blinking LED lighting of various colors.

In another example, providing the simplified communications device may include, for example, requesting an application for the simplified communications device. Further, in another example, providing the first application and the second application may each include making the respective application available for download.

While the invention has been described by reference to various specific embodiments, it should be understood that numerous changes may be made within the spirit and scope of the inventive concepts described. Accordingly, it is intended that the invention not be limited to the described embodiments, but will have full scope defined by the language of the following claims.

What is claimed is:

1. A simplified communications device, comprising:
a housing;
a plurality of keys coupled to the housing, each key corresponding to a different status message of a care recipient;
a first memory situated within the housing and storing a first application;
a processor situated within the housing, the processor configured to, in conjunction with the first application, cause the first application on the first computing device to send a status message corresponding to one of the plurality of keys, wherein the simplified communications device is capable of a wireless connection to the first computing device associated with the care recipient; and
wherein based on one of the plurality of keys being pressed, the status message is automatically sent from the first application executing on the first computing device to a second application executing on a second computing device associated with a caregiver.

2. The communications device of claim 1, wherein the plurality of keys comprises four keys.

3. The simplified communications device of claim 1, wherein the wireless connection uses a short-range wireless communications protocol.

4. The simplified communications device of claim 3, wherein the short-range wireless communications protocol comprises one of Bluetooth, Wi-Fi, a combination of low-power connectivity and a high-speed connection, near-field communication (NFC), radio frequency identification (RFID), low-power wide area network (LPWAN), ultra-wideband (UWB) and low-rate wireless personal area network (LR-WPAN).

5. The simplified communications device of claim 1, wherein the first application and the second application are a same application.

6. The simplified communications device of claim 1, wherein the second application is stored on a second memory associated with the second computing device.

7. The simplified communications device of claim 6, wherein each of the first memory and the second memory comprises one of an integrated memory and a removable memory.

8. The simplified communications device of claim 1, further comprising at least one of a different color or grayscale for each of the plurality of keys, descriptive Braille on or adjacent to each of the plurality of keys and descriptive text on or adjacent to each of the plurality of keys.

9. The simplified communications device of claim 1, wherein the plurality of keys comprise a plurality of physical keys.

10. The simplified communications device of claim 1, wherein the plurality of keys comprise one or more capacitive touch screens.

11. A method of communicating a status of a person, the method comprising:
providing a simplified communications device to a care recipient, wherein the simplified communications device comprises:
a housing;
a plurality of keys coupled to the housing, each key corresponding to a different status message of a care recipient;
a first memory situated within the housing and storing a first application; and
a processor situated within the housing, the processor configured to, in conjunction with the first application, cause the first application on the first computing device to send a status message corresponding to one of the plurality of keys, wherein the simplified communications device is capable of a wireless connection to the first computing device associated with the care recipient;
providing a second application stored in a second memory associated with a second computing device associated with a caregiver; and
based on one of the plurality of keys being pressed, automatically sending a status message corresponding to the one of the plurality of keys from the first application executing on the first computing device to the second application executing on the second computing device.

12. The method of claim 11, wherein the plurality of keys comprises four keys.

13. The method of claim 11, wherein the wireless connection uses a short-range wireless communications protocol.

14. The method of claim 13, wherein the short-range wireless communications protocol comprises one of Bluetooth, Wi-Fi, NearLink, near-field communication (NFC), radio frequency identification (RFID), Low-Power Wide Area Network (LPWAN), ultra-wideband (UWB) and low-rate wireless personal area network (LR-WPAN).

15. The method of claim 11, wherein the first application and the second application are a same application.

16. The method of claim 11, wherein the second application is stored on a second memory associated with the second computing device.

17. The method of claim 16, wherein each of the first memory and the second memory comprises one of an integrated memory and a removable memory.

18. The method of claim 11, further comprising at least one of a different color or grayscale for each of the plurality of keys, descriptive Braille on or adjacent to each of the plurality of keys and descriptive text on or adjacent to each of the plurality of keys.

19. The method of claim 11, wherein the plurality of keys comprise a plurality of physical keys.

20. The method of claim 11, wherein the plurality of keys comprise one or more capacitive touch screens.

21. The method of claim 11, wherein providing the simplified communications device comprises providing an application for the first computing device.

22. The method of claim 21, wherein providing the first application and providing the second application each comprises making the respective application available for downloading.
